(19) United States
(12) Patent Application Publication
(10) Pub. No.: US 2007/0208363 A1
(43) Pub. Date: Sep. 6, 2007

(54) Device and Method of Minimally Invasive Tattooing and Tattoo Removal

(76) Inventor: Shui T. Lai, Encinitas, CA (US)

Correspondence Address: JACKSON & CO., LLP, 6114 LA SALLE AVENUE, #507, OAKLAND, CA, 94611-2802, US

(21) Appl. No.: 11/682,241

(22) Filed: Mar. 5, 2007

Related U.S. Application Data
(60) Provisional application No. 60/767,126, filed on Mar. 5, 2006.

(51) Int. Cl. A61B 17/34

(52) U.S. Cl. 606/186

(57) ABSTRACT

A method of tattooing includes removing substantially an epidermis cell layer, without substantially removing a corresponding basal cell layer, at a skin area where a tattoo is desired. A tattoo is applied on or near the basal cell layer of the skin area. Ink from the tattoo has an enhanced tendency to migrate into a dermis layer of the skin area due to the removing of the epidermis cell layer and applying of the tattoo instead to the basal cell layer.

### PRIORITY

This application claims the benefit of priority to U.S. provisional patent application No. 60/767,126, filed Mar. 5, 2006, which is hereby incorporated by reference.

### BACKGROUND

Currently tattoos are made with a handheld instrument that includes a motorized needle. Ink is delivered into the skin by piercing the needle into the skin, one point at a time. The needle penetration can be as deep as 3-5 mm, i.e., all the way to major vascular vessels. The process is long, painful, and may even result in scaring. The choice of colors is limited, typically to 4-5 colors, since it is impractical to ink the needle with a new color between needle pricks. Another disadvantage of conventional tattooing processes is that blood born infections, such as hepatitis, are commonly incurred as a result of use of unsantized needles.

Tattoo removal is a significant surgical procedure that typically involves multiple laser treatment sessions, e.g., up to 8 or more sessions is not uncommon. It is costly in time and money. The results are often not satisfactory. Healing may involve infections and complications.

In U.S. Pat. Nos. 6,013,122 and 6,814,760, which are hereby incorporated by reference, methods of modifying tattoo ink are provided. However, both patents teach encapsulating the ink particles to form a sufficiently large unit comparable to large ink particles used in typical tattooing processes, such that it may not be dispersed away after they are delivered into the skin. While the methods attempts to address problems associated with tattoo removal, they still involve the conventional method of delivering the ink unit using needles. Therefore, inherent with these methods are the disadvantages of using needles in tattoos.

### SUMMARY OF THE INVENTION

A method of tattooing is provided including removing substantially an epidermis cell layer, without substantially removing a corresponding basal cell layer, at a skin area where a tattoo is desired. A tattoo is applied on or near the basal cell layer of the skin area. Ink from the tattoo has an enhanced tendency to migrate into a dermis layer of the skin area due to the removing of the epidermis cell layer and applying of the tattoo instead to the basal cell layer.

The method may include applying a first polymer to the ink. The polymer and the ink cross-link to form polymerized ink. The polymerized ink may have at least one dimension smaller than 100 nm.

The polymerized ink may be mixed with a second polymer. The first polymer then cross-links with the second polymer when both the polymerized ink and the second polymer are within the surface of the skin area. The mixing may occur prior to the applying of the tattoo. The mixing may include applying the second polymer at or near the basal layer. The mixing of the polymerized ink and the second polymer then occurs within the skin area, and the applying of the second polymer occurs prior to or after the applying of the tattoo, or both.

The method may also include driving the polymerized ink into the skin. The driving may include use of diffusion, and/or applying heat, micro-vibrations, ultrasound, an electric field and/or a magnetic field.

The applying of the tattoo may include depositing the tattoo ink onto a decal in accordance with a tattoo design, and applying the decal to the skin area. An energy source may be applied to the location of tattoo ink within the skin area, thereby facilitating cross-linking among the tattoo ink, the first polymer, the second polymer, and collagen fibers in the dermis layer. The energy source may include UV, visible light, infrared, heat, or electron beam radiation, or combinations thereof.

The applying of the tattoo may include applying a first polymer to tattoo ink, wherein the first polymer and the ink cross-link to form polymerized ink. The polymerized ink may then be applied as a tattoo decal on or near the basal cell layer of the skin area. The skin area may then be pierced with a needle. Tattoo ink may be delivered in a region substantially localized within a 0.5 to 2 mm depth region within the skin area. The piercing depth of the needle may be controlled using an applanation plate which is coupled to a tattooing instrument that houses the needle. The applanation plate may be in contact with the skin and be at least momentarily stationary before each needle piercing. The applanation plate may provide a fixed frame of reference for the needle to thereby control the depth. The tattooing instrument may include a driving mechanism that moves the needle along the axis of the needle.

In another method involving applying the first polymer to tattoo ink which cross-link to form polymerized ink, the polymerized ink may be processed electrically to strip off or attach one or more electrons thereto. The processed polymerized ink may be applied on or near the basal cell layer of the skin area. A first electrode may be disposed over the skin area of the tattoo and a second electrode may contact a body part away from the skin area of the tattoo. An electric field may be generated between the electrodes to drive the processed polymerized ink into the skin.

In another method involving applying the first polymer to tattoo ink which cross-link to form polymerized ink, the polymerized ink includes a magnetic structural unit. The polymerized ink is applied on or near the basal cell layer, and a magnetic field is applied to the skin area of the tattoo to drive the ink into the skin.

A virtually painless tattooing method is provided including applying a tattoo decal on a skin area where a tattoo is desired. The skin area is pierced with a needle whose penetration depth is controlled. Tattoo ink is delivered substantially localized within an approximately 0.5 mm depth region of the skin area. The delivering of the tattoo ink may utilize an instrument that couples with the needle. A driving mechanism may move the needle along the axis of the needle. An applanation plate may be disposed at a location relative to the needle along the direction of the needle axis which is adjustable to control an extension of the needle beyond a surface of the plate which is put in contact with the skin during tattooing.

A tattooing instrument may include a needle, a driving mechanism that moves the needle along the axis of the needle, and an applanation plate coupled to the tattoo instrument. The plate location is adjustable relative to the needle along the direction of the needle axis to control an extension of needle beyond the plate surface which is put in contact with the skin during tattooing.

A laser skin removal device includes a laser generating laser pulses having wavelengths of 380 nm or less and pulse durations of 200 nanoseconds or less and/or pulse durations of 5 picoseconds or less and energy densities in the range of 5 micro-joules to 100 micro-joules per 25 square microns. The laser may include an excimer laser, or a titanium sapphire, Nd:YAG, Nd:YLF, or Alexandrite laser. The wavelengths may be the fundamental wavelengths, and/or second third, fourth or fifth harmonics of the fundamental laser wavelengths may be generated.

A tattoo decal may include a substrate with multiple microscopic inkwells for receiving tattoo ink. The tattoo ink may be ejected from an ink reservoir onto the substrate. One or more of the inkwells may be generated by etching at additive centers of the substrate and/or by etching while an electric field is applied across the substrate.

A device for producing a removable tattoo at a skin area includes tattoo ink processed with a first polymer to form polymerized ink, wherein at least one dimension of the polymerized ink is about 100 nm or smaller. A second polymer is delivered with the polymerized ink into a dermis layer of the skin area. An energy irradiation source for generating UV, visible, infrared, heat, or an electron beam, or combinations thereof, irradiates the skin area facilitating cross-linking in the dermis layer between the polymerized ink and the second polymer. Cross-linking is to occur in the dermis layer between the polymerized ink, second polymer and collagen fibers. The tattoo ink may have a particle size of approximately 100 nm or less. The tattoo decal may include a tattoo design produced with the polymerized ink. A delivery mechanism may include a needle for piercing the skin area permitting diffusion of the polymerized ink through the skin. The instrument may apply heat, micro-vibrations, an electric field or a magnetic field, or combinations thereof, to drive the polymerized ink into the skin.

A method of producing a removable tattoo includes providing tattoo ink including ink particles having a particle size of approximately 100 nm or less. The ink particles are polymerized with a first polymer. The polymerized ink is delivered into a dermis layer of a skin area where a tattoo is desired. A second polymer is delivered into the dermis layer. Cross-linking is facilitated among the polymerized ink, the second polymer, and collagen fibers in the dermis layer.

The facilitating may be provided by UV, visible, infrared, or electron beam irradiation, or combinations thereof. A tattoo design may be produced in a decal using the polymerized ink. The decal is then applied to the skin area. Multiple microscopic inkwells may be produced in a substrate of the decal for receiving the polymerized ink. The delivering of the polymerized ink and/or the second polymer may include piercing the skin with a needle permitting diffusion of the polymerized ink through the skin, and applying heat, micro-vibrations, ultra sound, an electric field or a magnetic field, or combinations thereof, to drive the polymerized ink into the skin.

A method of removing a tattoo, includes selecting a tattoo produced by a method described above or below herein. The, a pulsed laser is selected for generating laser pulses at a wavelength that is absorbed by cross-linked ink units in the dermis layer of the skin area including the tattoo to be removed. A laser irradiation energy density is set to about five joules per square centimeter or less for breaking polymer bonds with ink particles, and releasing the ink particles from polymerized ink units. The laser irradiation energy density may be set to about one joule per square centimeter or less.

An ultra sound tattooing device is provided including tattoo ink and a decal including a tattoo design. An ink jetting instrument is for jetting tattoo ink onto the decal. A contact tip is connected to an ultrasound transducer for generating ultrasound vibrations at the contact tip. The contact tip contacts an area within the tattoo decal, thereby driving the tattoo ink into the skin. The contact tip may contact an area within the tattoo decal having at least one of its dimensions ranging from 1 mm to 10 cm.

### BRIEF DESCRIPTION OF THE DRAWINGS

FIG. 1 schematically illustrates a skin area, e.g., where a tattoo may be desired.

FIGS. 2a-2f illustrates stages in a tattooing process in accordance with a preferred embodiment.

FIG. 3 schematically illustrates an instrument for producing a tattoo in accordance with a preferred embodiment.

FIG. 4 is a flow diagram illustrating a method in accordance with a preferred embodiment.

FIG. 5 illustrates use of ultrasound in a tattooing method.

### DETAILED DESCRIPTION OF THE PREFERRED EMBODIMENTS

A method of delivering ink into the skin in tattooing is provided. The present method has considerable advantage in that it involves relatively less time, and relatively less pain than conventional processes. In a preferred embodiment, a tattooing method is virtually painless. In another embodiment, color and gray density can be the same level as that of modern computer printers, i.e., several million colors and a gray level of 10-12 bits, both of which are beyond the capability of the current tattooing methods. Furthermore, the tattoos created using methods according to certain embodiments can be removed with less treatment, e.g., fewer treatment sessions, using a laser.

### Removal of Epidermis Layer

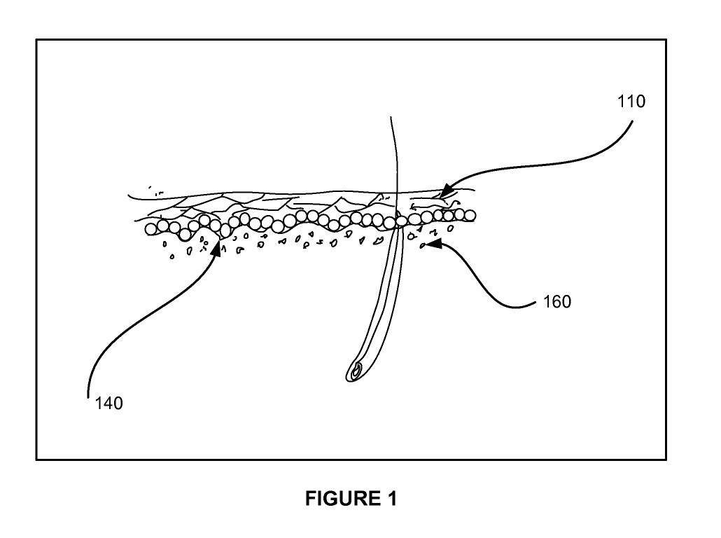

A skin area, e.g., where it may be desired to place a tattoo, includes an epidermis layer 110, as illustrated in FIG. 1, which includes mostly older skin cells that are densely packed to protect the skin from external intrusions of bacteria and other germs. This layer 110 serves particularly well to repel fluids including water and maintaining body hydration.

In one embodiment, a microdermabrasion is performed to remove the epidermis up to or near a basal cell layer 140. The microdermabrasion uses fine sand particles or other ablation method to remove the superficial skin cells 110, including a horny layer, transition layer and granular layer. Alternatively, and as an embodiment a laser dermablation is provide herein. An UV laser such as an excimer laser is used to remove the epidermis layer cell. Excimer lasers have the advantage of high precision to thereby remove the cells layer by cell layer. An ArF excimer laser operating at 193 nm, e.g., interacts with tissue by vaporizing the cells, by absorption of high energy photons at 193 nm or similar wavelength (such as other excimer laser UV wavelengths such as 248 nm, or UV wavelength photons generated by another laser medium, including up-conversion processes and harmonic generation processes to arrive at the preferred wavelengths of less than 380 nm. In the case of excimer lasers operating at 193 nm, the laser pulses may be set at about 20 mJ per square cm and each laser pulse may ablates less than or about 0.25 microns per laser pulse without leaving excessive heat at the dermis layer (excimer microdermablation). Preferably, another type of laser that generates a laser wavelength 250 nm or shorter can be used. The short UV wavelength with sufficiently short pulse duration of less than 100 nanoseconds provides efficient and precise skin removal at a thin layer of about 0.2 to 0.1 microns in thickness for each the laser pulse. Other laser sources are: the fundamental, second harmonics, third harmonics, fourth and fifth harmonics may be used from a list of laser media, e.g., as described in the following. For example, a fourth harmonic of titanium sapphire laser, fifth harmonic of Nd:YAG or Nd:YLF, and fourth harmonic of Alexandrite laser which each generate UV wavelengths. Various laser wavelengths can also be generated by optical parametric oscillation and amplification methods, in which two or more photons can combine their energies to provide shorter wavelength photons.

In another embodiment, a skin ablation laser is chosen which has an ultra-short pulse duration of about 5 picoseconds or less. The laser pulses can be generated using a mode-locked oscillator and amplifier system, in laser media such as Nd:YLF, ND:YAG, Nd:Glass, Ti:sapphire, or another laser media which has a laser gain band width of about 1 nm or wider. The ultra short pulse nature of the laser concentrates the pulse energy into short time durations and hence a high peak power. The ablation threshold is reduced in terms of laser energy density to 5 to 100 micro-joules per (5 microns)² or 25 square microns, yet maintaining a high peak power to break down the skin tissue. The ablation depth can be precisely controlled to about less than one micron per laser pulse, hence a precise skin removal.

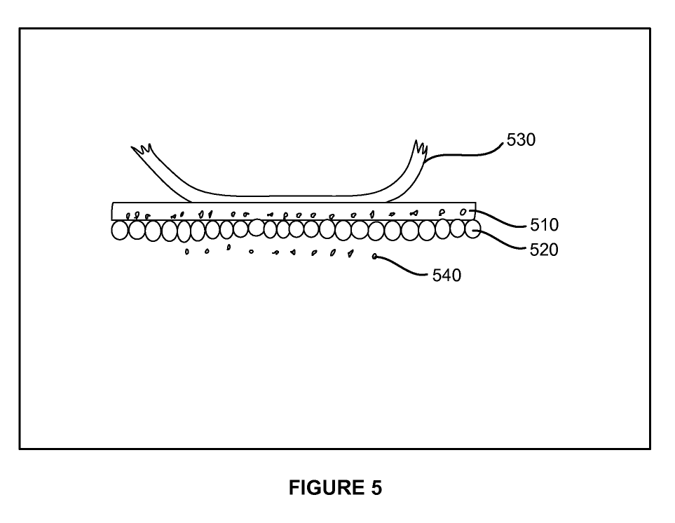

Advantages of performing epidermis removal include (a) reducing the resistance of ink penetration into the dermis layer of the skin, and (b) reducing the applicable penetration depth of the ink. The exposed basal cell layer 140 is a single cell layer, therefore ink particles 160 do not have to penetrate too deeply to reach the dermis layer where the ink particles 160 are to be delivered. The thickness of the basal cell layer does not vary significantly throughout the skin and is in the range of about 10-15 microns. Ink particles 160 may pass through the basal cell layer by diffusion, and may be localized in a region right next to basal cells as illustrated at FIG. 1. Ink penetration may also be encouraged by other means including heat, micro-vibration including the use of ultra-sound, electric filed, magnetic field and/or a needle with a well controlled penetration depth. More description on the use of ultra sound or vibration method is provided with reference to FIG. 5 below. Descriptions are provided in the following examples.

### Specially Formulated Inks and Anchoring with Collagen Fibers

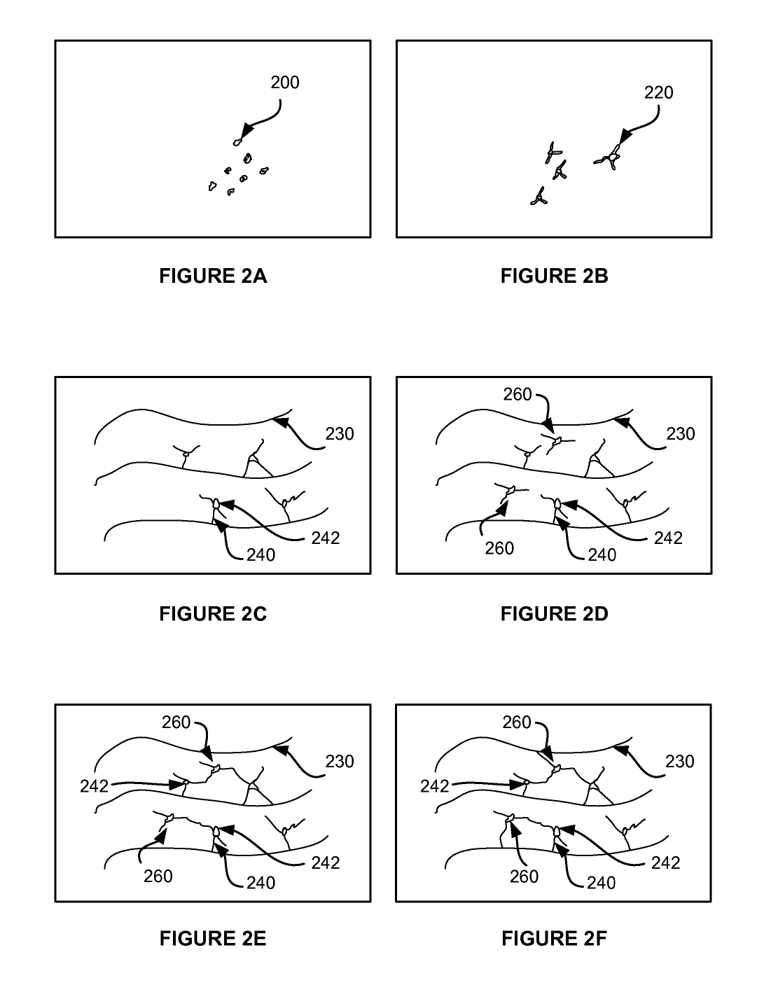

In another embodiment, specially formulated inks are used. As illustrated in FIG. 2A, chromatic components 200 are prepared with chemical processes that crystallize at a controlled environment that control the particle size, such that the chromatic particles are preferably less than one micron in dimension, and more preferably in a range between 2 nanometers to 200 nanometers (nm). There are FDA approved dyes and food colors, all of which may be used for the formulation of tattoo ink. A list of examples can be found in a table in U.S. Pat. No. 6,814,760 which is incorporated here in its entirety by reference.

The chromatic components are then processed to cause the particles to react with a polymer, or monomers, or low functional polymers to form polymerized inks 220, as illustrated at FIG. 2B. These polymer units 220 are made to remain small in at least one dimension, preferably less than 100 nm such that it can diffuse through the basal cell layer. Heat and micro vibration such as ultra sound may be applied to the polymerized ink to encourage the skin penetration process. More details on the use of vibration or ultra sound is described with reference to FIG. 5 below.

Secondly, the polymer component modifies the inorganic chromatic component that includes metal or oxides, into an organic compound. After its introduction into the skin and underneath the basal layer, the polymerized ink is then made to cross-link 240 with collagen fibers 230 directly, as illustrated in FIG. 2C. An intermediate polymer 260 or a second polymer unit 260 may be introduced as illustrated at FIG. 2D. Upon cross-linking of the polymerized inks 242 with the second polymer units 260, an extended polymerized structure is formed as illustrated at FIG. 2E. That structure is in turn cross linked to the collagen fibers 230 as shown in FIG. 2F. Whether by direct cross-linking or by cross-linking through intermediate or second polymer units 260, the process stabilizes the ink particles 200 in the dermis layer.

Another feature of the inks is that the second polymer system may be mixed with the ink component such that cross-linking does not occur between the second polymer and the polymerized ink prior to their delivery into the skin. By not polymerizing the ink outside of the skin, the ink component is kept to a smaller dimension. This facilitates diffusion into the skin. The second polymer, after it is delivered into the dermis, together with the chromatic components, are then made to cross-link under UV irradiation, or other energy sources such as short visible wavelength light, or infrared radiation, heat or other thermal sources, and/or an electron beam irradiation source. Cross-linking occurs between the polymerized ink and the collagen fibers, and between the second polymer component and collagen fibers, and between the polymerized ink and the second polymer units. The end result is that the ink is attached to the collagen fiber and it becomes immobilized when it is engulfed by macrophage cells, thereby making the tattoo permanent.

The types of polymers and polymer systems that can be used as the first polymer to bind the ink particles can be selected from a list comprising: thiol, ene, or many of the low functional polymers. The polymerized ink so formed preferably has substantially the same size of the small ink particle to start with. The purpose of polymerizing the ink particle is not to enlarge the size of the ink particle unit at this point, rather to enable cross linking of the ink particles with either the collagen fibers in the dermis layer, or the second polymer units after both of them are introduced inside the dermis layer. The polymerization with the second polymer unit or the collagen fibers around the ink particle is to build the overall size of the first polymerized ink particle to render it immobile in the dermis layer.

The second polymer unit may include: thiols, enes, acetates, celluloses, polyethylenes, methyl/ethyl celluloses, hydroxyls, glycols, acrylates, fatty acids/alcohol, wax, siloxy, fluoromethacrylates and combinations of one or more of the mentioned basic polymer structures, in mono-, di-, or poly-forms. Other polymers useful for this purpose can be found in U.S. Pat. No. 6,013,122, which is incorporated here in its entirety by reference.

### Tattoo Design in Specially Prepared Decal

In certain embodiments, an outline of a tattoo is traced onto the skin area using a carbon paper or other tattoo drawing transfer medium. The process can tend to be prone to human errors. Most tattoos are limited to pictures of single objects like a star, flag, dagger, dragon, devil, etc. Conventional tattooing methods are often limited by the lack of choice of colors and gray levels, therefore they are not able to produce sufficiently life-like tattoos. In another embodiment, an inkjet printer and laser printer method are used to first generate a life-like color decal or transfer, using the special ink as described earlier. With the present method, tattoo design is much more versatile. The tattoo design can be from photographs, printings, and most other art forms.

In another embodiment, the tattoo design can be deposited directly on the skin, a direct write method rather than first transferring the ink to a decal, or other tattoo design transfer.

Traditional decal tattoos, such as Henna tattoos, are not permanent. The design may be deposited onto the surface of the epidermis, and this layer is to be shed away in about 2-3 weeks, and hence the temporary tattoo is also gone. In the present method, the polymerized ink, with or without second polymer units, is fed into an ink reservoir of a printer. Here the printer shall refer to any means of depositing color ink, regardless of the mechanism of how the ink is ejected. In an embodiment, ejection mechanism comprises a piezoelectric actuator that propels microscopic ink drops to a target area. In another embodiment, the ejection mechanism comprises pressurized air that is premixed with ink droplets that are produced through an atomizer, and the ink droplets are then forced out of a nozzle, which is orientated to shoot the droplet to a predetermined target area. The various ink jetting methods and their device configurations as disclosed in U.S. Pat. No. 6,836,371 and US published patent application 2005/0046957, which are incorporated by reference herein in their entirety.

The substrate of the decal comprises a water penetrable sheet material such that when a tattoo graphic separates from the decal, it is transferred to a skin area that has been properly prepared (epidermis layer 110 has been removed) as described earlier with reference to FIG. 1. In a further embodiment, the substrate of the decal is coated with a layer of porous polymer that, in turn, holds the microscopic ink droplets, when the ink is deposited onto the porous layer by a ink printer. In one embodiment, the pores are microscopic in the range of 10 nanometers to 200 microns. The formation of pores is controlled by an additive introduced in the polymer coating material. An etching process is applied to etch away an additive component thereby leaving microscopic voids. Alternatively, the additive component is used to initiate an etching while an electric or magnetic field is applied across the coating layer. Many other etch methods may be used which have been developed in the field of nanotechnology to create nanotubes in carbon silicon, and substrates used thereof, and are understood by those skilled in the art. With these “pores”, microscopic ink reservoirs (inkwells) may be created across the substrate; which in turn enables increasing of ink volume to be delivered in a tattoo.

One advantage of the microscopic inkwell embodiment is that water is preferably not used as a carrier for the ink, although alternatively water may be used. In one embodiment, the ink is stored in separate inkwells with or without dilution with water. A sealant layer is then applied over the inkwells to hold the ink in place until it is to be delivered. The overcoat layer can be made of a water-soluble material. When the decal is applied to a wet skin surface, the protective overcoat dissolves and allows the ink to flow out of the reservoirs.

No limitation is to be implied here as to when the polymerized ink and the second polymer are mixed or come into the vicinity of each other. In one case, the second polymer is first mixed with the polymerized ink, and then the mixture is used to produce a tattoo decal. In another embodiment, the polymerized ink and the second polymer can be applied to the skin in two separate steps, and the mixing may occur inside the skin. Also, no limitation is to be implied as to whether a tattoo decal with the polymerized ink is applied to the skin first, followed by applying the second polymer to the same region of the skin, or vice versa.

### Tattooing with a Needle and an Applanation Plate

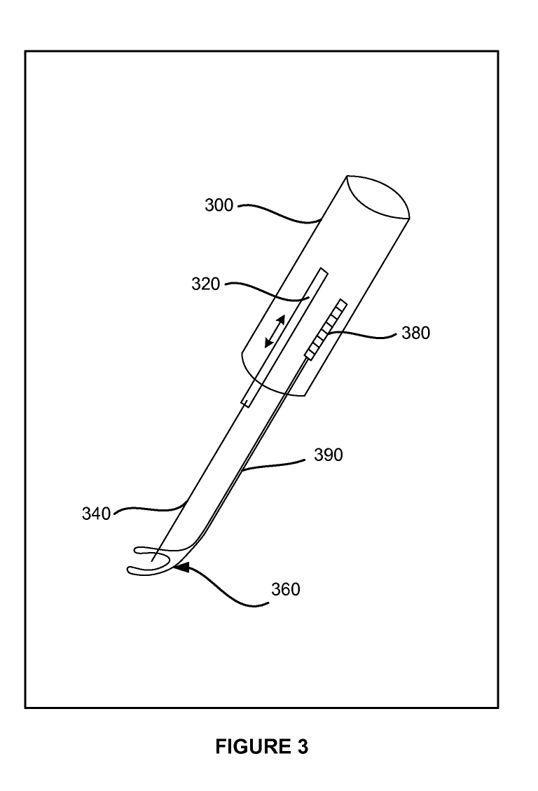

In one embodiment, one would use a needle to enable ink delivery into the dermis layer. An advantageous feature in accordance with another embodiment is the inclusion of an applanation plate with the needle, which provides a depth reference from the surface of skin. The applanation plate may be attached to the hand piece of a motorized tattoo needle assembly and may be placed in contact with the skin when tattooing is performed. The applanation plate can be in a shape similar to that of a footplate of a sewing machine, which includes a slit opening for the needle to go through to pierce the skin. The applanation plate may be used as a reference affixed to the needle instrument and may be used to set the penetration depth of the needle. A schematic diagram of a needle instrument with an applanation plate in accordance with a one embodiment is shown in FIG. 3. The tattoo instrument 300 includes a needle 340 or a needle block including multiple needles 340, which is attached to a needle shaft 320. The needle shaft 320 moves along a track that is in the direction of the piercing axis of the needle 340. Shaft movement may be induced by a magnetic field generated in a wire coil through which alternating current is fed. Other means of driving the needle may include a motor, resonance coil, piezoelectric actuator, or other means of generating oscillating movement as understood by those skilled in the art. An example of an applanation plate 360 is also illustrated in FIG. 3. It has a preferably rigid extension arm 390 connecting to the instrument 300. The plate extension distance can be adjusted with a fine threaded screw drive 380 that pushes against the extension arm 390 of the applanation plate 360. The desired range of needle penetration depth is between 0.1 mm to 3 mm. In a preferred embodiment, the applanation plate is set for a needle penetration depth in the range of 0.2 to 0.5 mm from the top surface of basal cell layer. An advantage of having an applanated needle is that it provides uniformity in ink deposition depth, as compared to ink deposition at random depths such as when a tattoo is made with a conventional handheld tattoo machine, which can often result in an inferior color saturation and image contrast to the method of this embodiment. Another advantage of a shallow ink deposition into the skin is that less pain is induced.

In another embodiment, the applanated needle assembly may include more than one needle. The needles may be set in a row, with 10 or more needles arranged in a linear arrangement. The separation distance between needles is set between 50 microns to 2000 microns (2 mm). There is no upper limit on how far apart the needles are disposed. A lower limit may be related to the dimension of the needle diameter. The preferred needle separation is approximately one time or larger of the needle diameter at the desired depth in the skin. For example, if the needle diameter at 0.2 mm below the basal layer is 75 microns, then the needles separation may be set at 75 microns apart or farther. In another embodiment, the needles are arranged in two dimensions, for example, 50 needles by 10 needles rectangular, or 50 by 50 square blocks. These examples do not limit the embodiment, either to increase or to decrease the number of needles in any one dimension. The needles can also be arranged in a parallelepiped formation, circular, solid circle, solid oval shapes, or other shape. One application of the applanation needle assembly is to produce multiple ink entry points in one application, pushing down the needle set into a predetermined depth into the skin, as determined by the depth setting of the applanation plate. The linear or two dimensional needle blocks are then translated to a new location to produce additional ink entry points, until a sufficient number of ink entry points has been created to cover the tattoo image on the decal.

### Improving Conventional Tattooing

In one embodiment, an otherwise conventional needle tattoo instrument is incorporated with an applanation plate. Regular tattoo ink may be used in an applanated needle instrument. This instrument provides a more precise piercing depth when the advantageous applanation plate is used, pierce after pierce, delivering the ink to a precise depth level. By depositing tattooing ink at a shallow depth, the method substantially reduces pain, and can be virtually painless.

### Pain Reduction Due to Shallow Needle Penetration

There are several advantages of setting the applanation plate for a needle extension of less than 0.5 mm, which is shallower than conventional techniques. First, the ink is deposited in a narrowly defined layer; and the tattoo image is more vivid in color, and in contrast. Because a nerve network is denser in and below the mid dermis, and is rare right underneath the basal layer, the ink delivery has decreased pain, and may be virtually painless.

### Driving Tattoo Inks into Skin Layer Using Electric and Magnetic Field

In another embodiment, the ink particle together with the cross-linked polymer is charged either positively or negatively. The charging can be made passing the ink compound between a positive and negative electrode while the compound is activated with strong electric current, or bombarded with an electron beam, or exposed to a selected light beam that can induce a single or multi-photon transition to release or adsorb an electron in the outer orbital. The ink particle and the cross-linked polymer can also be selected from a class of material that has a magnetic dipole component, and that will interact with a magnetic field.

In one embodiment, electrically charged ink or magnetized ink is applied to form a tattoo decal. The decal is applied to the skin. A protective cover coating may be dissolved, and ink may be in contact with the basal cell layer. A positive electrode may be applied over the decal and a negative electrode may be attached to the other side of an arm, e.g., as in the case of making a tattoo on the arm. An electric field or magnetic field may be applied to drive the ink into the dermis layer.

In another embodiment, the needle block described above is used to apply an electric field. In this case, the penetration depth can be very shallow, in the range of 30-100 microns. Once the ink entry points are made, the electric field is applied to drive the ink through the entry points. Alternatively, the needles are set to barely penetrate the basal layer. The electric field may be applied through the tips of the needles.

Due to a small surface area at the tip of the needle, the local electric strength around the tip region is strong. Therefore, using needles, instead of a larger area electrode can substantially enhance the driving force.

The embodiments for delivering the ink into the skin are also applicable to delivering the second polymer into the skin. The delivering of the second polymer can be selected from: piercing the skin with a needle permitting diffusion of the polymerized ink through the skin, and applying heat, micro vibrations, including use of ultra sound, and/or applying electric and/or magnetic fields to drive the polymerized ink into the skin.

### Tattoos that are Permanent Yet Removable

Among the methods of tattoo removal, such as salabrasion, acid etching, full thickness skin removal, dermabrasion, and sand paper, laser tattoo removal is a preferred method that results in relatively low scar formation. In a typical laser tattoo removal, an intensive laser pulse with laser energy density of about 10 Joules per cm² is applied to break down the ink particle into smaller pieces. Some of those are cleaned up and removed by macrophage cells, a normal skin defense function. However, a substantial portion of the tattoo often remains. A minimum of typically four repeated laser treatment sessions might be needed in a conventional tattoo removal process. Up to 8-10 sessions is not uncommon. The costs increase with each additional treatment session.

In the ink embodiments described earlier, the ink particles are processed and selected for their small dimensions of about 100 nm or smaller. If an ink particle is in the dermis layer by itself, without cross-linking via a pre-processed polymer or a second polymer unit, the dirty removal process of the macrophage cells would have removed the ink particle, and the tattoo would not be permanent. However, cross-linking among the ink, first polymer, polymerized ink, the second polymers and the collagen fibers in the dermis layer renders the ink particle immobile from the macrophage cells.

To remove a tattoo using these specially prepared inks, and when tattoo design is delivered into the skin in accordance with a preferred embodiment, an advantageous laser tattoo removal process is provided in accordance with another embodiment. The laser energy density is reduced to less than the typical 10 Joules per cm² used with ordinary tattoo removal processes, and can be reduced to even less than 5.0, 2.0 or 1.0 Joules per cm² in alternative embodiments depending on the particular chemistry and interaction of the first and second polymers, the polymerized ink and collagen fibers, and the detailed method. In a particular embodiment, the energy density is advantageously reduced to about 0.5 Joules per cm² or less. In the conventional tattoo removal processes, the large ink particles are broken down into smaller pieces by a photo-disruption process. In those processes, the laser energy density exceeds those at the level of electronic breakdown, at over 10 joules per cm². On the other hand, photo-absorption is preferably used to initiate the polymer bond breaking process in this embodiment. In photo-absorption the energy level starts to build up immediately at any incident laser energy level, until weak organic bonds being broken at significantly less energy. Another benefit of using lower laser energy density is that, either the laser output energy can be reduced, or a beam of greater total energy can be enlarged in area, i.e., to cover a larger treatment area, hence an increased treatment efficiency for the laser. Furthermore, the tattoo ink may be confined in a shallower region, in a layer of about 0.5 mm in depth, for example, as compared to a large depth region of 2-4 mm for a conventional tattoo. The rate of absorption of the laser energy by the embedded ink may also be more uniform for the entire tattoo. Hence the removal is more efficient, and takes fewer treatment sessions and/or temporally less treatment. Thirdly, using less laser energy per pulse, the laser pulses produce less heat, hence less induced inflammation. Taken together with reduced treatment sessions, the chance of producing scars is substantially reduced. Also, the treatment time is reduced which reduces the costs, and pain, but with a healthier skin and quicker recovery.

### Tattooing Method

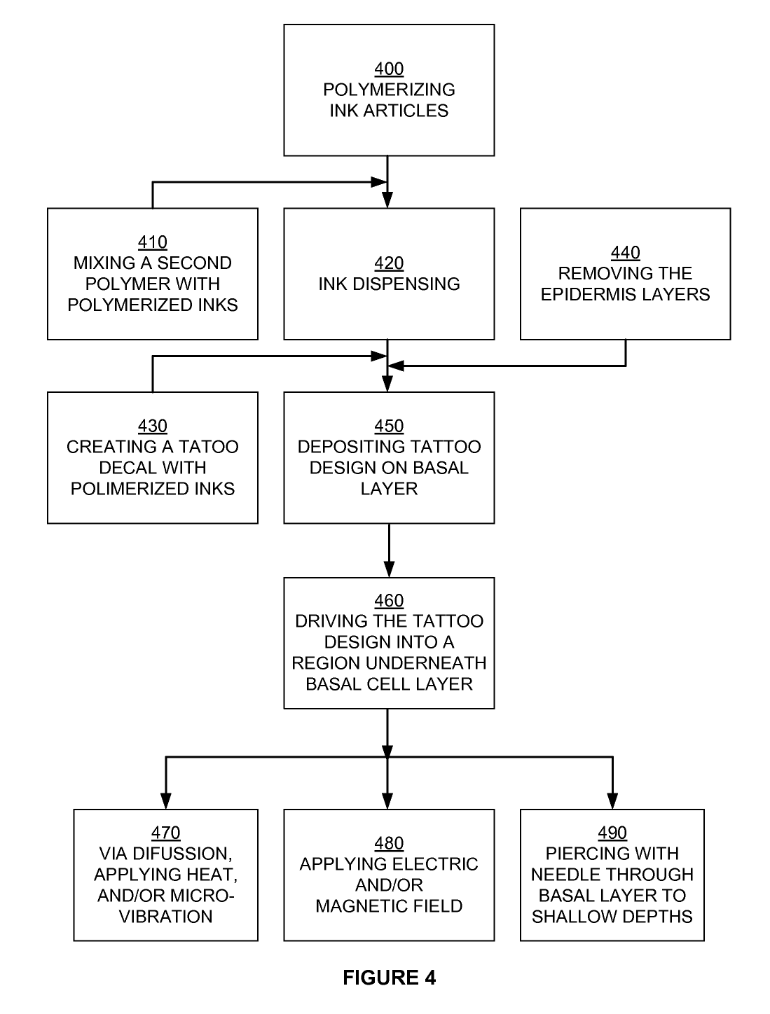

An exemplary tattooing method is shown in the block diagram of FIG. 4. In Box 400, ink particles are refined and or filtered to control the particle size to less than 100 nm in at least one dimension. The refined ink is then polymerized with low functional polymers, to enable easy cross linking with collagen fiber in the dermis layer, or a second polymer as described earlier.

In Box 410, second polymer units are mixed with polymerized ink. The mixed polymer and ink are then used to make tattoos.

In Box 420, the polymer inks alone, or after they have been mixed with the second polymers are transferred to an ink dispensing device, which can be an inkjet printer, or other jetting devices as previously described.

In Box 430, a tattoo design is formed on a decal or some transfer medium, using the polymerized inks, and optionally mixed with the second polymers.

In Box 440, the skin area is prepared for tattooing preferably by dermabrasion using sand particles, or laser derm-ablation using a laser with precise tissue removal capability. The epidermal layer is removed substantially and preferably without inflicting substantial damage to the basal cell layer of the skin area.

In Box 450 of FIG. 4, the tattoo design is deposited onto or substantially near or proximate to or close to the basal layer. The depositing method includes, but is not limited to, transfer of the polymerized ink from the decal, and direct-writing of a design from an ink jetting device to the skin.

In Box 460, the tattoo inks are driven into the dermis layer. Preferably the inks are localized in a shallow region of about 0.5 mm beneath the basal layer, however, this is not a limiting factor and the depth may be controllably varied, preferably using an applanation plate that is adjustably coupled with a needle and/or needle drive mechanism.

In Box 470, the method of driving the tattoo inks is by diffusing the ink into the skin. Additional aids such as heat, or micro-vibrations such as ultra sound, may be applied to encourage the ink penetration. An illustration of the use of sound wave or ultrasound is provided at FIG. 5. The micro vibration contact tip 530 is in contact with a decal or other tattoo ink transfer medium 510, in which a tattoo design has been formed with polymerized inks in the decal or porous material as described earlier. Sound waves are generated through a transducer (not shown) which converts electrical energy into vibrations. The transducer is mechanically attached to the contact tip. The shape of the contact surface of the contact tip can be flat or curved in one or two or three dimensions. In the example of FIG. 5, the contact surface of the tip is flat in the center and has curved edges. The operator may slide the contract surface over a desired area by a sliding or rolling motion to make good contact of the tip to the tattoo decal and the skin surface underneath. Vibrational energy is transmitted from the transducer to the contact tip 530, and then to the ink particles. The energized ink 540 migrates, passing the basal cell layer 520 into the dermis layer, as illustrated at FIG. 5. The contact tip 530 can have a contact surface ranging from 1 mm to 10 cm in at least one dimension, covering a portion or covering substantially the entire intended tattoo area, although no limitation is implied on the area dimension or the shape of the contact tip 530.

In Box 480, in the case of electrically-charged polymerized inks, an electric field is applied to speed the ink penetration through the basal layer.

In Box 480, in the case of polymer inks constructed with magnetic properties, a magnetic field is applied to drive the ink into the skin through the basal layer.

In Box 490, a method involving a needle is used. Preferably, the method uses a penetration depth which is well controlled by using an applanating plate, such as a “foot plate”, such that the needle pierces to a substantially constant depth which is ideally to be barely through the basal layer, e.g., 0.2 to 0.5 mm.

The present invention is not limited to the embodiments described above herein, which may be amended or modified without departing from the scope of the present invention as set forth in the appended claims, and structural and functional equivalents thereof.

In methods that may be performed according to preferred embodiments herein and that may have been described above and/or claimed below, the operations have been described in selected typographical sequences. However, the sequences have been selected and so ordered for typographical convenience and are not intended to imply any particular order for performing the operations.

In addition, all references cited above herein, in addition to the background and summary of the invention sections, as well as US Published patent applications nos. 2005/0046957 and 2005/0061198, and U.S. Pat. Nos. 6,013,122, 6,841,760 and 6,341,831, are hereby incorporated by reference into the detailed description of the preferred embodiments as disclosing alternative embodiments and components.

I claim:

1. A method of tattooing, comprising:
removing substantially an epidermis cell layer, without substantially removing a corresponding basal cell layer, at a skin area where a tattoo is desired; and
applying a tattoo on or near the basal cell layer of said skin area;
wherein ink from the tattoo has an enhanced tendency to migrate into a dermis layer of said skin area due to the removing of the epidermis cell layer and applying of the tattoo instead to the basal cell layer.

2. The method of claim 1, further comprising applying a first polymer to the ink wherein the polymer and the ink cross-link to form polymerized ink, and wherein the polymerized ink has at least one dimension smaller than 100 nm.

3. The method of claim 2, further comprising mixing with the polymerized ink a second polymer, wherein the first polymer cross-links with the second polymer when both the polymerized ink and the second polymer are within the surface of the skin area.

4. The method of claim 3, wherein the mixing occurs prior to the applying of the tattoo.

5. The method of claim 3, wherein the mixing comprises applying the second polymer at or near the basal layer, and wherein the mixing of the polymerized ink and the second polymer occurs within the skin area, and the applying of the second polymer occurs prior to or after the applying of the tattoo, or both.

6. The method of claim 2, further comprising driving the polymerized ink into the skin, wherein the driving utilizes diffusion, or application of heat, micro-vibrations, ultrasound, an electric or field, or combinations thereof.

7. The method of claim 1, wherein the applying comprises depositing the tattoo ink onto a decal in accordance with a tattoo design, and applying the decal to the skin area.

8. The method of claim 7, further comprising applying energy source to the location of tattoo ink within the skin area, and thereby facilitating cross-linking among the tattoo ink, the first polymer, the second polymer, and collagen fibers in the dermis layer, and wherein the energy source comprises UV, visible light, infrared, heat, or electron beam radiation, or combinations thereof.

9. The method of tattooing of claim 1, wherein the applying comprises:
applying a first polymer to tattoo ink, wherein the first polymer and the ink cross-link to form polymerized ink;
applying the polymerized ink as a tattoo decal on or near the basal cell layer of said skin area;
piercing the skin area with at least one needle; and
delivering tattoo ink in a region substantially localized within a 0.5 to 2 mm depth region within the skin area.

10. The method of claim 9, further comprising controlling a piercing depth of the needle using an applanation plate which is coupled to a tattooing instrument that houses the needle, wherein the applanation plate is in contact with the skin and is at least momentarily stationary before each needle piercing, and wherein the applanation plate provides a fixed frame of reference for the needle to thereby control said depth.

11. The method of claim 10, wherein the tattooing instrument comprises a driving mechanism that moves the needle along the axis of the needle.

12. A tattooing instrument, comprising:
a. at least one needle;
b. a driving mechanism that moves the needle along the axis of the needle;
c. an applanation plate coupled to the tattoo instrument, wherein the plate location relative to the needle along the direction of the needle axis is adjustable to control an extension of needle beyond the plate surface which is put in contact with the skin during tattooing.

13. The method of tattooing of claim 1, wherein the applying comprises:
applying a first polymer to tattoo ink, wherein the first polymer and the ink cross-link to form polymerized ink,
processing electrically the polymerized ink to strip off or attach one or more electrons thereto,
applying the processed polymerized ink on or near the basal cell layer of the skin area;
applying a first electrode over the skin area of the tattoo and a second electrode to a body part away from the skin area of the tattoo;
generating an electric field between the electrodes to drive the processed polymerized ink into the skin.

14. The method of tattooing of claim 1, wherein the applying comprises:
applying a first polymer to tattoo ink, wherein the first polymer and the ink cross-link to form polymerized ink that comprises at least one magnetic structural unit;
applying said polymerized ink on or near the basal cell layer; and
applying a magnetic field to the skin area of the tattoo to drive the ink into the skin.

15. A virtually painless tattooing method, comprising:
applying a tattoo decal on a skin area where a tattoo is desired;
piercing the skin area with at least one needle;
controlling a needle penetration depth; and
delivering tattoo ink substantially localized within an approximately 0.5 mm depth region of the skin area.

16. The method of claim 15, wherein the delivering of the tattoo ink utilizes an instrument that couples with the needle, and comprises a driving mechanism that moves the needle along the axis of the needle, and an applanation plate disposed at a location relative to the needle along the direction of the needle axis which is adjustable to control an extension of the needle beyond a surface of the plate which is put in contact with the skin during tattooing.

17. A laser skin removal device, comprising a laser generating laser pulses having wavelengths of 380 nm or less and pulse durations of 200 nanoseconds or less.

18. The laser skin removal device of claim 17, comprising a laser selected from: an Excimer laser, a titanium sapphire, Nd:YAG, Nd:YLF, or Alexandrite laser, wherein the wavelengths are the fundamental wavelengths, or generating a second a third, fourth or fifth harmonic of the fundamental laser wavelengths, or combinations thereof.

19. A laser skin removal device, comprising a laser generating laser pulses having pulse durations of 5 picoseconds or less and energy densities in the range of 5 micro-joules to 100 micro-joules per 25 square microns.

20. A tattoo decal, comprising:
a substrate comprising plurality of microscopic inkwells for receiving tattoo ink; and
tattoo ink ejected from an ink reservoir onto the substrate.

21. The tattoo decal of claim 20, wherein one or more of the inkwells are generated by etching at additive centers of the substrate.

22. The tattoo decal of claim 20, wherein one or more of the inkwells are generated by etching while an electric field is applied across the substrate.

23. A device for producing a removable tattoo at a skin area, comprising:
tattoo ink processed with a first polymer to form polymerized ink, wherein at least one dimension of the polymerized ink is about 100 nm or smaller;
a second polymer which is delivered with the polymerized ink into a dermis layer of the skin area;
an energy irradiation source for generating UV, visible, infrared, heat, or an electron beam, or combinations thereof, for irradiating the skin area facilitating cross-linking in the dermis layer between the polymerized ink and the second polymer.

24. The device of claim 23, wherein cross-linking is to occur in the dermis layer between the polymerized ink, second polymer and collagen fibers.

25. The device of claim 23, wherein the tattoo ink has a particle size of approximately 100 nm or less.

26. The device of claim 23, further comprising a tattoo decal comprising a tattoo design produced with the polymerized ink.

27. The device of claim 23, further comprising delivering means including a needle for piercing the skin area permitting diffusion of the polymerized ink through the skin, and an instrument for applying heat, micro-vibrations, an electric field or a magnetic field, or combinations thereof, to drive the polymerized ink into the skin.

28. A method of producing a removable tattoo, comprising:
providing tattoo ink including ink particles having a particle size of approximately 100 nm or less;
polymerizing the ink particles with a first polymer;
delivering the polymerized ink into a dermis layer of a skin area where a tattoo is desired;
delivering a second polymer into the dermis layer;
facilitating cross-linking among the polymerized ink, the second polymer, and collagen fibers in the dermis layer.

29. The method claim 28, wherein the facilitating is provided by UV, visible, infrared, or electron beam irradiation, or combinations thereof.

30. The method of claim 28, further comprising producing a tattoo design in a decal using the polymerized ink and applying the decal to the skin area.

31. The method of claim 30, further comprising producing a plurality of microscopic inkwells in a substrate of the decal for receiving the polymerize d ink.

32. The method of claim 28, wherein the delivering of the polymerized ink comprises piercing the skin with a needle permitting diffusion of the polymerized ink through the skin, and applying heat, micro-vibrations, ultra sound, an electric field or a magnetic field, or combinations thereof, to drive the polymerized ink into the skin.

33. The method of claim 28, wherein the delivering of the second polymer comprises piercing the skin with a needle permitting diffusion of the second polymer through the skin, and applying heat, micro-vibrations, ultra sound, an electric field or a magnetic field, or combinations thereof, to drive the second polymer into the skin.

34. A method of removing a tattoo, comprising:
selecting a tattoo produced by the method of claim 28;
selecting a pulsed laser for generating a plurality of laser pulses at a wavelength that is absorbed by cross-linked ink units in the dermis layer of the skin area including the tattoo to be removed;
setting a laser irradiation energy density to about five joules per square centimeters or less, to break polymer bonds with ink particles, and release the ink particles from polymerized ink units.

35. The method of claim 34, wherein the laser irradiation energy density is set to about one joule per square centimeter or less.

36. A method of removing a tattoo, comprising:
selecting a tattoo produced by the method of claim 1;
selecting a pulsed laser for generating a plurality of laser pulses at a wavelength that is absorbed by cross-linked ink units in the dermis layer of the skin area including the tattoo to be removed;
setting a laser irradiation energy density to about five joules per square centimeters or less, to break polymer bonds with ink particles, and release the ink particles from polymerized ink units.

37. The method of claim 36, wherein the laser irradiation energy density is set to about one joule per square centimeter or less.

38. A sound wave tattooing device, comprising:
tattoo ink;
a decal comprising a tattoo design;
an ink jetting instrument for jetting tattoo ink onto the decal; and
a contact tip connected to one or both of a sound wave or ultrasound transducer for generating ultrasound vibrations at the contact tip;
wherein the contact tip is further for contacting an area within the tattoo decal, and for thereby driving the tattoo ink into the skin.

39. The device of claim 38, wherein the contact tip is for contacting an area within the tattoo decal having at least one of its dimensions ranging from 1 mm to 10 cm, and a contact surface of the contact tip has a flat or curved shape in one or more dimensions.

40. The device of claim 38, further comprising an epidermis removal device for removing substantially an epidermis of a tattoo area without substantially removing basal cells in the tattoo area.
